(19) United States
(12) Patent Application Publication
(10) Pub. No.: US 2020/0060742 A1
(43) Pub. Date: Feb. 27, 2020

(54) Access device for providing access to a fixation element fixed to tissue

(71) Applicant: Spinal Developments Pty Ltd, North Sydney, New South Wales (AU)

(72) Inventors: Gregory James ROGER, Milsons Point, New South Wales (AU); Davor Drago SARAVANJA, East Lindfield, New South Wales (AU)

(21) Appl. No.: 16/344,105

(22) PCT Filed: Oct. 24, 2017
(86) PCT No.: PCT/AU2017/051161

§ 371 Date: Apr. 23, 2019

(30) Foreign Application Priority Data
Oct. 24, 2016 (AU) 2016904317

(51) Int. Cl. A61B 17/88; A61B 17/064; A61B 17/84; A61B 17/86; A61B 17/34; A61B 17/68

(52) U.S. Cl. CPC A61B 17/8872; A61B 17/0642; A61B 17/846; A61B 17/86; A61B 2017/90; A61B 17/8875; A61B 17/8863; A61B 17/686; A61B 17/3423

(57) ABSTRACT

An access device for providing access to a fixation element is disclosed. The access device includes an elongate body, the elongate body having one or more first openings at a first end of the elongate body, a second opening at a second end of the elongate body, and a conduit extending between the first and second openings. The access device also includes a connection portion at the second end of the elongate body for connecting to the fixation element. When the connection portion is connected to the fixation element, an access pathway is provided from the one or more first openings of the elongate body, through the conduit of the elongate body, to a contact portion of the fixation element.

### CROSS-REFERENCE TO RELATED APPLICATIONS

The present application claims priority to Australian provisional patent application no. 2016904317, the content of which is hereby incorporated by reference.

### TECHNICAL FIELD

The present disclosure relates to surgery, such as orthopaedic surgery, in which a fixation element such as a screw is fixed to bone or other tissue.

### BACKGROUND

During surgery, such as orthopaedic surgery, fixation elements such as bone anchors, fiducial markers, pedicle screws or similar devices are often fixed to bone or other tissue, either permanently or temporarily, using an insertion tool such as a screw driver. Typically, the fixing is performed while directly observing the fixation element and the insertion tool. The direct observation is performed in an attempt to check for safe placement of the fixation element, to allow the ready relocation of the fixation element if required, and to reduce the likelihood that the fixation element becomes unknowingly disconnected from the introduction tool and becomes lost in tissues during surgery.

However, in some instances such direct observation is not possible. For example, during minimally invasive surgery (MIS), also known as keyhole surgery, very small openings in tissue are used to access the sites at which fixation elements are located. The tissue surrounding the access openings can therefore obscure the insertion sites, the screws and/or portions of insertion tools. When direct observation is not possible, safe and reliable placement of the fixation elements may be difficult and there may be an increased risk that disconnection between the fixation element and the introduction tool occurs without a surgeon knowing, or in a such a way that loss of the fixation element in tissue becomes likely.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

### SUMMARY

According to one aspect of the present disclosure, there is provided an access device for providing access to a fixation element, the fixation element having a fixation portion for fixing to body tissue and a contact portion connected to the fixation portion, the access device comprising:

an elongate body, the elongate body having one or more first openings at a first end of the elongate body, a second opening at a second end of the elongate body, and a conduit extending between the first and second openings; and

a connection portion at the second end of the elongate body for connecting to the fixation element,

wherein, when the connection portion is connected to the fixation element, an access pathway is provided from the one or more first openings of the elongate body, through the conduit of the elongate body, to the contact portion of the fixation element.

The fixation element may be any fixation element configured to fix to tissue and which has a fixation portion and a contact portion. For example, the fixation element may be a screw, plug or nail that includes a shaft and a head, or a staple that includes at least two shafts extending from a head. In general, the fixation portion of the fixation element may therefore be at least one shaft and the contact portion may be a head connected to the at least one shaft. The shaft may be a threaded shaft and/or may include one or more barbs, for example. The head may have a greater diameter than a diameter of the shaft. However, the fixation element need not necessarily include a head and shaft. The fixation element may be a toggling means such as an Endobutton™, for example, which includes a suture providing a fixation portion and a toggle element providing a contact portion.

When the connection portion is connected to the fixation element, some or all of the contact portion, e.g. the head, of the fixation element may locate in the conduit. Moreover, the connection portion may extend underneath the contact portion of the fixation element and partially or entirely surround the fixation portion, e.g. the shaft, of the fixation element. The contact portion may be held within the conduit while that fixation portion extends through the connection portion. In some embodiments, the contact portion of the fixation element may be integrated with the connection portion of the elongate body during manufacture (such as during injection moulding of the elongate body). Thus, the contact portion of the fixation element may be substantially permanently fixed to the elongate body.

The connection portion may comprise a rim defining the second opening of the elongate body, which rim may extend underneath the contact portion of the fixation element and partially or entirely surround the fixation portion of the fixation element. Additionally or alternatively, the connection portion may comprise a narrowing of the conduit of the elongate body adjacent the second opening of the elongate body, which narrowing may extend underneath the contact portion of the fixation element and partially or entirely surround the fixation portion of the fixation element. Additionally or alternatively, the connection portion may comprise one or more transverse portions that extend transversely to the direction of elongation of the elongate body, and the one or more transverse portions may extend underneath the contact portion of the fixation element and partially or entirely surround the fixation portion of the fixation element. Additionally or alternatively, the connection portion may comprise elastic material that can have a temporary dimensional change so as to, for instance, release or recapture the contact portion of the fixation device.

At least one of the one or more first openings of the elongate body may have a larger diameter than a diameter of the contact portion, e.g. the head, of the fixation element and the second opening of the elongate body may have a smaller diameter than the diameter of the contact portion, e.g. the head, of the fixation element. Nonetheless, the second opening of the elongate body may have larger diameter than a diameter of the fixation portion, e.g. the shaft, of the fixation element. Thus, at least one of the first openings may be large enough for the entire fixation element, including the contact portion and the fixation portion, to be passed through the first opening so that the fixation element can be inserted into the conduit via the first opening. However, the second opening may be too small for the contact portion to be extended through the second opening, but may be large enough for the fixation portion to be extended through the second opening. Thus, after inserting the fixation element into the conduit via the first opening, the fixation portion may be extended through the second opening.

When the connection portion of the access device is connected to the fixation element, the elongate body may be adapted to receive one or more insertion tools that extend via the one or more first openings, through the conduit and that are operable to cause insertion of the fixation element into the tissue. Additionally or alternatively, when the connection portion of the access device is connected to the fixation element, the elongate body may be adapted to receive one or more navigation probes that extend via the one or more first openings through the conduit to probe the position of the fixation element.

The elongate body may be tubular. The elongate body may be a sleeve. Where more than one first opening is provided, the elongate body may have a Y-shaped configuration or otherwise. The elongate body may therefore bifurcate or trifurcate to allow multiple points of access to the second opening via the one or more first openings.

The elongate body may comprise depth markings to provide an indication of the depth that the elongate body extends into tissue. The elongate body may be formed of stiff material or a pliable material. The elongate body may formed from suitable biocompatible materials such as plastic or metal. The material may be radiopaque or radiolucent. The elongate body may be formed of elastic material allowing temporary dimensional change so as to, for instance, release or recapture the contact portion, e.g., the head, of the fixation device. The length of the elongate body may be adjustable. For example, the elongate body may be configured to be trimmed to adjust the length of the elongate body.

The elongate body may be configured to be breakable to remove the access device from connection with the fixation element, e.g. when the fixation element is to be permanently fixed to body tissue. The elongate body may comprise one or more frangible portions or lines of weakness to enable breaking of the elongate body.

In general, the access device may aid in the safe and secure placement of the fixation element during surgery, the ready finding of the fixation element during surgery, and the easy retrieval of the fixation element at the end of surgery, for example.

In addition to use in receiving and guiding tools or probes, the access device may provide a visual indicator of the location of the fixation element. Moreover, the access device may be used to facilitate the passing of sutures and securing tapes through its length, for instance during shoulder reconstructive surgery.

The access device may be used with fixation elements adapted to secure bone, e.g. to secure the femoral end of an ACL graft, so that the fixation element can be re-acquired during surgery if adjustments need to be made.

The access device may also be used as an electrical sensor or connector, by having one or more electrodes and/or conductive elements extending along or through walls of the elongate body or extending through the conduit. In one embodiment, for example, the elongate body may comprise a conductive mesh embedded in its wall. In another embodiment, for example, the elongate body may comprise an electrode array along an outer surface of the elongate body. By functioning as an electrical connector or sensor, the access device may be used in the detection of nearby nerves, for instance, during endoscopic carpal tunnel surgery or otherwise. Moreover, the access device may also act as an antennae for interaction with an external computer or electromagnetic frequency device to aid in ease of tracking. The access device may also have trackable infra-red optical markings allowing the position of the fixation element to which it is connected to be tracked in real time. Moreover, the elongate body may include one or more light sources to provide illumination to, for example, the fixation element. The one or more light sources may comprise fibre optic elements and/or LEDs attached to or integrated into the walls of the elongate body, for example.

The access device may be useable as a tissue retractor for visualisation of intervening tissues though which the access device passes, as well as tissues to which it is attached via the fixation element. So that it may better act as a tissue retractor, particularly if material forming the elongate body is pliable, the elongate body may include one or more stiffening elements that are integrated into walls of the elongate body and/or that are placed in or on the elongate body as a separate step.

In some embodiments, the access device may be used as a rail for passing an ultrasound probe through tissue. The probe may probe in a direction perpendicular to the direction of elongation of the elongate body of the access device, allowing for imaging of the tissues along the path of the access device in the body. Imaging or interrogation modalities other than ultrasound may also use the access device as a known and repeatable path of usage.

In one aspect of the present disclosure there is provided surgical apparatus comprising: a fixation element for fixing to body tissue, the fixation element having a fixation portion for fixing to body tissue and a contact portion connected to the fixation portion; and an access device according to the preceding aspect, the connection portion of the access device being connected to the fixation element.

In some embodiments, the surgical apparatus may comprise a tool for cutting or breaking open the access device to release the access device from the fixation element.

In another aspect of the present disclosure there is provided a method of using the access device or surgical apparatus as described in accordance with preceding aspects.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### BRIEF DESCRIPTION OF DRAWINGS

By way of example only, embodiments of the present disclosure are now described with reference to the accompanying drawings in which:

FIGS. 1a and 1b show bottom and top oblique views, respectively, of an access device according to an embodiment of the present disclosure;

FIG. 2a to 2c show a top oblique transparent view, a side view, and a side transparent view, respectively, of the access device of FIG. 1a in conjunction with a fixation element, and FIG. 2d shows an enlargement of Area B in FIG. 2c;

FIG. 3 shows a side view of the access device and fixation element of FIG. 2b, where the fixation element is fixed to bone tissue using an insertion tool extending through the access device;

FIGS. 4a and 4b show side views of the access device and fixation element of FIG. 2b, the fixation device being fixed to bone tissue, where the length of the access device is reduced; and

FIG. 5a shows a top oblique view of an access device according to another embodiment of the present disclosure used in conjunction with a releasing tool for releasing the access device from a fixation element; and FIG. 5b shows an enlargement of Area A in FIG. 5a.

### DESCRIPTION OF EMBODIMENTS

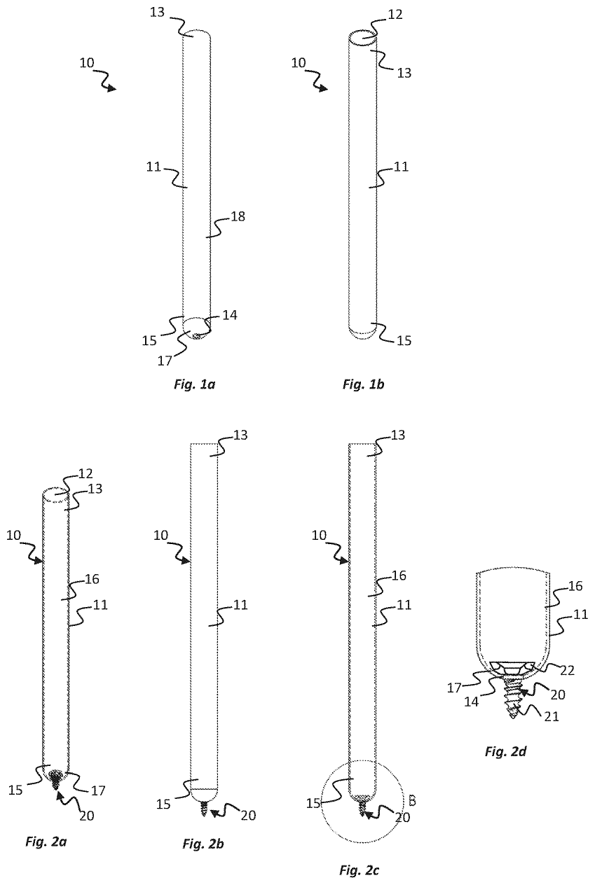

An access device 10 according to an embodiment of the present disclosure is illustrated in FIGS. 1a and 1b. The access device 10 includes an elongate body 11 having a first opening 12 at a first end 13 and a second opening 14 at a second end 15. A conduit 16 extends between the first and second openings 12, 14 and first and second ends 13, 15 in the direction of elongation of the elongate body 11 (see also FIGS. 2a and 2c).

As illustrated in FIGS. 2a to 2d, the access device 10 is used in conjunction with a fixation element 20. In this embodiment, the fixation element 20 has fixation portion provided by a shaft 21 for extending into body tissue, such as bone tissue, and a contact portion provided by a head 22 that is connected to the shaft 21. The shaft 21 is a threaded shaft adapted to be screwed into tissue in this embodiment.

In this embodiment, the fixation element 20 is a fiducial screw (fiducial marker) used as a point of reference when body tissue, to which the fiducial screw is fixed, is imaged using an external imaging system or otherwise. However, the access device 10 may be used with a variety of different fixation elements such as bone anchors, pedicle screws, polyaxial bone screws, toggling means such as Endobuttons™ or otherwise. The fixation elements may have in common at least a fixation portion, such as a shaft, for extending into or attaching to tissue, and a contact portion, such as a head, connected to the fixation portion. The contact portion may have a larger diameter than the fixation portion.

In this embodiment, the elongate body 11 comprises a cylindrical tube 18 extending from the first end 13 of the body 11 along most of the length of the elongate body 11, and a connection portion 17 at the second end 15 of the elongate body 11. The connection portion 17 is for connecting to the fixation element 20. The connection portion 17 is provided by a rim of the elongate body 11 that extends roughly transversely to the direction of elongation of the elongate body 11 and that defines the second opening 14. The diameter of the second opening 14 is smaller than the diameter of the first opening 12.

The diameter of the first opening 12 is large enough for the entire fixation element 20, including the shaft 21 and the head 22, to be passed through the first opening 12. The fixation element 20 can therefore be inserted into the conduit 16 via the first opening 12.

The diameter of the second opening 14 is too small for the head 22 to be extended through the second opening 14, but is large enough for the shaft 21 to be extended through the second opening 14. Thus, after inserting the fixation element 20 into the conduit 16 via the first opening 12, the shaft 21 can extend through the second opening 14 such that the connection portion 17 surrounds the shaft 21, while the underside of the head 22 abuts the inner surfaces of the connection portion 17, as illustrated in FIG. 2d, for example. The connection portion 17 in this embodiment includes curved inner surfaces to conform to curved surfaces of the underside of the head 22. Thus, particularly when the fixation element 20 is subsequently fixed to tissue, the access device 10 can be retained in position on the fixation element 20, the connection portion 17 of the access device being trapped between the underside of the head 22 of the fixation element 20 and the tissue.

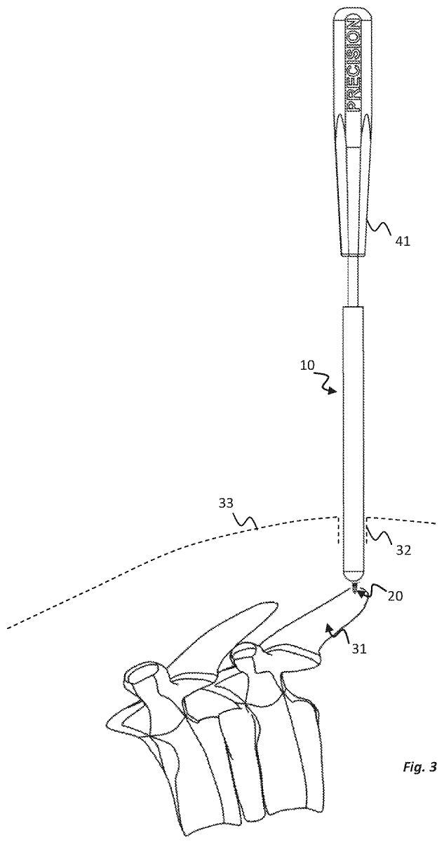

FIG. 3 shows a representation of the access device 10 retained in position on a fixation element 20 when the fixation element 20 is fixed to bone tissue 31. In this example, the bone tissue 31 is vertebral bone tissue. The access device 10 is arranged to extend through an incision 32 in body tissue 33 adjacent the bone tissue 31, e.g. muscle, fat and/or skin, generally illustrated using dotted lines in FIG. 3.

The access device 10 can therefore provide a clear access path for tools, instruments or other items to reach through body tissue 33 and interface with the fixation element 20, e.g., during a step of fixing of the fixation element 20 to bone tissue 31, a step of removing the fixation element 20 from bone tissue 31, and/or when carrying out procedures while the fixation element is fixed to the bone tissue 31. The tools or instruments can reach through the first opening 12 of the access device 10, through the conduit 16 and interface with the head 22 of the fixation element 20 inside the conduit 16. An example insertion tool is partially illustrated in FIG. 3, and is specifically a screw driver 41 that engages with and turns the head 22 of the fixation element 20 in order to drive the shaft 21 of the fixation element 20 into the bone tissue 31 and/or remove the shaft 21 from the bone tissue 31. Additionally or alternatively, a navigation probe may be extended through the conduit 16 to touch and therefore probe the position of the head 22 of the fixation device 20. The navigation probe may include infrared reflectors and may be used in conjunction with surgical navigation apparatus.

The access device 10 may therefore aid in the safe and secure placement of the fixation element 20 during surgery, the ready finding of the fixation element 20 during surgery, and the easy retrieval of the fixation element 20 at the end of surgery, for example.

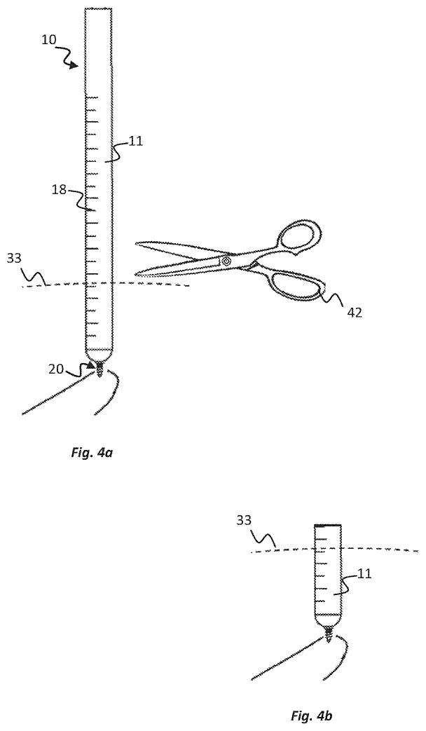

In one embodiment, as illustrated in FIG. 4a, the elongate body 11 of the access device 10 includes depth markings 18 to provide an indication of the depth to which the elongate body 11 extends into tissue 33.

The elongate body 11 is formed of a stiff material in this embodiment but, in alternative embodiments, all or part of the elongate body maybe formed of pliable material or otherwise. The elongate body 11 may be opaque or partially or entirely transparent. The elongate body 11 may formed from suitable biocompatible materials such as plastic or metal. The material may be radiopaque or radiolucent.

The protrusion of the access device 10 from the body tissue 33 may provide a useful visual indicator of the location of the fixation element 20. Nevertheless, the length of the elongate body 11 of the access device 10 can be adjusted to adjust the degree by which the access device projects from the body tissue 33. As illustrated in FIGS. 4a and 4b, for example, the length of the elongate body 11 can be adjusted by cutting the elongate body 11 using scissors 42 or another cutting tool such as a scalpel or knife.

In general, the access device 10 can be of sufficient length such that, once the fixation element 20 is secured, the open first end 12 of the access device presents to the outside of the incision 32, beyond the skin or other exposed tissue of the patient. However, to reduce any interference or obstruction caused by the access device 10 in the surgical field during surgery, while still maintaining a clear and easily accessible access path through the access device 10, a distal portion of the access device 10 can be removed by cutting, as discussed above.

If it is desired for the fixation element 20 to remain in the patient at the completion of surgery, the access device 10 may be released from the fixation element 20. The access device 10 may include one or more features to allow it to be released from the fixation element 20 while the fixation element 20 remains fixed to the body tissue. For example, the elongate body 11 of the access device 10 can include one or more frangible portions or lines of weakness to enable breaking of the elongate body 11.

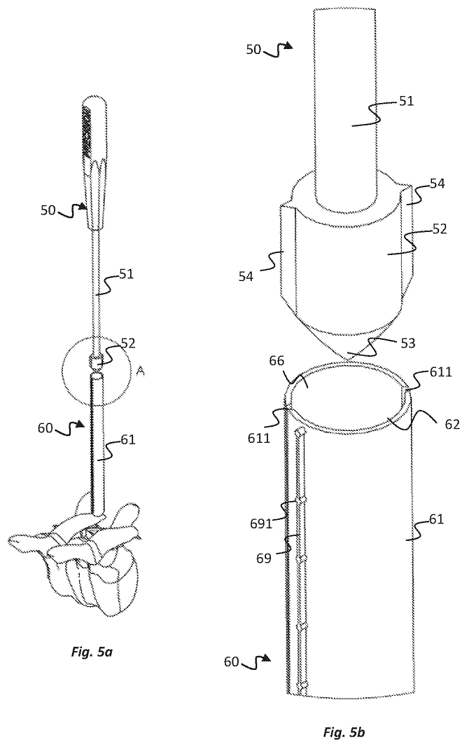

Referring to FIGS. 5a and 5b, for example, a releasing tool 50 may be used to release an access device 60 from a fixation element 20. The releasing tool 50 can cause breaking and/or cutting of the elongate body 61 of the access device 60. In this further embodiment, the access device 60 is generally configured in accordance with the access device 10 described above, but includes at least one line of weakness 611 (e.g. a tear or score line), and specifically two opposing lines of weakness 611 in this embodiment, that extend along a length of the inside of the wall that forms the elongate body 61. The releasing tool 50 has a shaft 51 with a cutting head 52 at one end that is configured to extend into the conduit 66 of the elongate body 61 via the first opening 62 of the elongate body 61. The cutting head 52 has a pointed end 53 and two cutting wings 54, which cutting wings 54 project outwardly form the cutting head and which are designed to cut through the wall of the elongate body 61 at the lines of weakness 611, as the cutting head 52 is extended through the elongate body 61, causing the elongate body 61 to be cut open. In alternative embodiments, no lines of weakness may be provided, but a similar cutting result may still be achieved. The cutting wings 53 may extend no further than the outer diameter of the elongate body 61, ensuring that they do not cause cutting of adjacent body tissue. As the cutting head 52 extends along the conduit 66, it may divide the elongate body 61 into two parts, enabling release of the access device 60 from the fixation element.

The access device 60 also includes an electrode array 69. The electrode array 69 includes a plurality of independently addressable electrodes 691 located intermittently along the length of the elongate body 61 on the outer surface of the wall that forms the elongate body 61. The electrode array 69 may be used to perform bioimpedance monitoring or other monitoring techniques at different tissue locations and depths.

In general, the access devices of the present disclosure can be used to provide electrical connection to the fixation element and/or surrounding body tissue, and/or to provide electrical sensing capabilities. The access devices may have one or more conductive elements extending along or through walls of the elongate body or extending through the conduit. In one embodiment, for example, the elongate body may comprise a conductive mesh embedded in its wall. By functioning as an electrical connector or sensor, the access device may be used in the detection of nearby nerves, for instance, during endoscopic carpal tunnel surgery or otherwise. Moreover, the access device may also act as an antennae for interaction with an external computer or electromagnetic frequency device to aid in ease of tracking. The access device may also have trackable infra-red optical markings allowing the position of the fixation element to which it is connected to be tracked in real time. Moreover, the elongate body may include one or more light sources to provide illumination to, for example, the fixation element. The one or more light sources may comprise fibre optic elements and/or LEDs attached to or integrated into the walls of the elongate body, for example.

Moreover, in some embodiments, the access devices may be useable as tissue retractors for visualisation of intervening tissues though which the access device passes, as well as tissues to which it is attached via the fixation element. So that an access device may better act as a tissue retractor, particularly if material forming the elongate body of the access device is pliable, the elongate body may include one or more stiffening elements that are integrated into walls of the elongate body and/or that are placed in or on the elongate body as a separate step.

In some embodiments, the access devices may be used as a rail for passing an ultrasound probe through tissue. The probe may probe in a direction perpendicular to the direction of elongation of the elongate body of the access device, allowing for imaging of the tissues along the path of the access device in the body. Imaging or interrogation modalities other than ultrasound may also use the access device as a known and repeatable path of usage.

It will be appreciated by persons skilled in the art that numerous variations and/or modifications may be made to the above-described embodiments, without departing from the broad general scope of the present disclosure. For example, the access devices may be permanently fixed to respective fixation elements, e.g. during manufacture. Further, more than one first opening may be provided to allow access for multiple tools, instruments and/or probes to the fixation element at the same time. When more than one first opening is provided, the elongate body may bifurcate or trifurcate, towards the first end of the device, for example. The present embodiments are, therefore, to be considered in all respects as illustrative and not restrictive.

1. An access device for providing access to a fixation element, the fixation element having a fixation portion for fixing to body tissue and a contact portion connected to the fixation portion, the access device comprising:
an elongate body, the elongate body having one or more first openings at a first end of the elongate body, a second opening at a second end of the elongate body, and a conduit extending between the first and second openings; and
a connection portion at the second end of the elongate body for connecting to the fixation element,
wherein, when the connection portion is connected to the fixation element, an access pathway is provided from the one or more first openings of the elongate body, through the conduit of the elongate body, to the contact portion of the fixation element.

2. The access device of claim 1, wherein:
the connection portion of the fixation element is a head and the fixation portion is a shaft extending from the head; and/or
the fixation element is a screw, plug, nail or staple.

3. The access device of claim 1, wherein, when the connection portion is connected to the fixation element, some or all of the contact portion of the fixation element is located in the conduit.

4. The access device of claim 1, wherein, when the connection portion is connected to the fixation element, the connection portion extends underneath the contact portion of the fixation element and partially or entirely surrounds the fixation portion of the fixation element.

5. The access device of claim 1, wherein the connection portion comprises:
a rim defining the second opening of the elongate body; or
a narrowing of the conduit of the elongate body adjacent the second opening of the elongate body.

6. (canceled)

7. The access device of claim 1, wherein the connection portion comprises one or more transverse portions that extend transversely to the direction of elongation of the elongate body at the second end of the elongate body.

8. The access device of claim 1, wherein at least one of the first opening of the elongate body has a larger diameter than a diameter of the connection portion of the fixation element.

9. The access device of claim 1, wherein the second opening of the elongate body has:
a smaller diameter than a diameter of the connection portion of the fixation element and a larger diameter than a diameter of the fixation portion of the fixation element.

10. (canceled)

11. The access device of claim 1, wherein the elongate body is tubular and/or is a sleeve.

12. (canceled)

13. The access device of claim 1, comprising a plurality of the first openings, and wherein the elongate body bifurcates or trifurcates towards the first end of the elongate body.

14. The access device of claim 1, wherein the elongate body comprises depth markings to provide an indication of the depth that the elongate body extends into tissue.

15. The access device of claim 1, wherein the elongate body is formed of stiff or pliable material.

16. (canceled)

17. The access device of claim 1, wherein the length of the elongate body is adjustable.

18. The access device of claim 17, wherein the elongate body is configured to be trimmed to adjust the length of the elongate body.

19. The access device of claim 1, wherein the elongate body is configured to be breakable to remove the access device from connection with the fixation element, the elongate body comprising one or more frangible portions or lines of weakness to enable breaking of the elongate body.

20. (canceled)

21. The access device of claim 1, wherein, when the connection portion of the access device is connected to the fixation element, the elongate body is adapted to receive:
an insertion tool that extends through the conduit and is operable to cause insertion of the fixation element into the tissue; or
a navigation probe that extends through the conduit to probe the position of the fixation element.

22-23. (canceled)

24. Surgical apparatus comprising:
a fixation element for fixing to body tissue, the fixation element having a fixation portion for fixing to the tissue and a contact portion connected to the fixation portion; and
an access device according to claim 1, the connection portion of the access device being connected to the fixation element.

25-27. (canceled)

28. The surgical apparatus of claim 24, further comprising a release tool for releasing connection between the access device and the fixation element, wherein the release tool is adapted to extend into the conduit of the elongate body and cause cutting or splitting of the elongate body.

29. The surgical apparatus of claim 24, further comprising a release tool for releasing connection between the access device and the fixation element, wherein the release tool is an elongate element that is extendable into the conduit and which has a larger diameter than inner surfaces of the elongate body surrounding the conduit such as to force apart or cut the elongate body.

30. The surgical apparatus of claim 24, wherein the fixation element is substantially permanently fixed to the access device during manufacture of the access device.
